(19) United States
(12) Patent Application Publication
(10) Pub. No.: US 2004/0176337 A1
(43) Pub. Date: Sep. 9, 2004

(54) Benzoquinolizine-2-carboxylic acid-containing compositions

(75) Inventors: Dilip G. Saoji, Aurangabad (IN); Rajendra N. Nagori, Aurangabad (IN); Milind C. Shukla, Aurangabad (IN); Sachin Bhagwat, Aurangabad (IN); Shrikant V. Gupte, Aurangabad (IN); Mahesh V. Patel, Aurangabad (IN); Rasendrakumar Jha, Mumbai (IN); Anil Kukreja, Mumbai (IN); Noel J. De Souza, Mumbai (IN)

Correspondence Address: Ladas & Parry, 26 West 61 Street, New York, NY, 10023, US

(73) Assignee: WOCKHARDT LIMITED

(21) Appl. No.: 10/749,933

(22) Filed: Dec. 31, 2003

(30) Foreign Application Priority Data
Dec. 31, 2002 (IN) 1170/MUM/2002

(51) Int. Cl. A61K 31/56; A61K 31/4745; A61K 31/203; A61K 31/07

(52) U.S. Cl. 514/171; 514/296; 514/559; 514/725

(57) ABSTRACT

This invention relates to topical compositions of an antibacterial benzoquinolizine-2-carboxylic acid, incorporated either as the single therapeutic ingredient in hitherto undescribed pharmaceutical compositions, or as an ingredient in novel combination with at least one agent selected from a retinoid, an antifungal agent, another antibacterial compound and/or a steroid/non-steroid anti-inflammatory agent, to processes for preparation of the compositions, to use of the compositions in preparation of a medicament, and to a method of therapeutic or prophylactic use of such a composition for the treatment of dermal, ophthalmic, otic and nasal infections, with or without attendant inflammation.

### FIELD OF THE INVENTION

This invention relates to topical compositions of an antibacterial benzoquinolizine-2-carboxylic acid, incorporated either as the single therapeutic ingredient in hitherto undescribed pharmaceutical compositions, or as an ingredient in novel combination with at least one agent selected from a retinoid, an antifungal agent, another antibacterial compound and/or a steroid/non-steroid anti-inflammatory agent, to processes for preparation of the compositions, to use of the compositions in preparation of a medicament, and to a method of therapeutic or prophylactic use of such a composition for the treatment of dermal, ophthalmic, otic and nasal infections, with or without attendant inflammation.

### BACKGROUND OF THE INVENTION

Topical compositions are useful for a wide range of dermal infection-originating disorders, ranging from those that are skin-related to those that are related to specific body parts, such as ophthalmic, otic and nasal disorders. The incidence and epidemiology of these different disorders is well documented in the scientific and patent literature.

The use of a benzoquinolizine-2-carboxylic acid antibacterial to treat infections represents the current state of the art in the field of dermal pharmaceutical compositions and methods of treatment. For example, a topical dermal composition containing the benzoquinolizine-2-carboxylic acid, RS-(±)-9-fluoro-6,7-dihydro-8-(4-hydroxypiperidin-1-yl)-5-methyl-1-oxo-1H,5H-benzo[i,j]quinolizine-2-carboxylic acid, is marketed by Wockhardt Limited, India under the name NADOXIN™(Nadifloxacin 1%) Cream.

Although RS-(±)-9-fluoro-6,7-dihydro-8-(4-hydroxypiperidin-1-yl)-5-methyl-1-oxo-1H,5H-benzo[i,j]quinolizine-2-carboxylic acid, from chemical considerations, belongs to the class of benzoquinolizine-2-carboxylic acids, it is classified from considerations of its antibacterial mode of inhibition of one or both of the essential Type II DNA topoisomerase enzymes viz. DNA gyrase and Topoisomerse IV, and its common quinolone core moiety, as a quinolone antibacterial, with its given name analogous in terminology to drugs like ciprofloxacin and levofloxacin.

RS-(±)-9-fluoro-6,7-dihydro-8-(4-hydroxypiperidin-1-yl)-5-methyl-1-oxo-1H,5H-benzo[i,j]quinolizine-2-carboxylic acid has also been utilized in other dermal antibiotic compositions:

ACUATIM™ Cream, Otsuka Pharmaceuticals, Japan

ACUATIM™ Lotion, Otsuka Pharmaceuticals, Japan

In the field of ophthalmic pharmaceutical formulations and methods of treatment, the current state of the art embraces the use of quinolone antibiotics such as ciprofloxacin, ofloxacin, norfloxacin, and lomefloxacin as outlined in U.S. Pat. No. 6,395,476 and WO 0018404 (PCT /US 99/22625), the contents of which are incorporated herein by reference.

Among benzoquinolizine-2-carboxylic acids reported to have therapeutically and/or prophylactically useful antibiotic, in particular antibacterial effect are those illustratively disclosed in the following patents and patent applications, each of which is individually incorporated herein by reference:

U.S. Pat. No. 4,399,134;

U.S. Pat. No. 4,552,879;

U.S. Pat. No. 6,514,986;

U.S. Pat. No. 6,608,669

IN 188847;

Chem. Pharm. Bull. 44 (1996), 642-5;

U.S. application Ser. No. 09/566,875 filed on May 8, 2000;

PCT Application No. PCT/IN00/00054 filed on May 8, 2000;

U.S. application Ser. No. 09/640,947 filed on Aug. 17, 2001;

PCT Application No. PCT/IN00/00111 filed on Nov. 2000;

PCT Application No. PCT/IN01/00097 filed on May 3, 2001 (WO 0185095);

PCT Application No. PCT/IN01/00100 filed on May 8, 2001 (WO 185728);

U.S. Pat. No. 6,664,267;

PCT Application No. PCT/IN02/00123 filed on May 28, 2002 (WO 03099815);

PCT Application No. PCT/US02/12790 filed on Apr. 24, 2002 and U.S. Application No. 2002/0187193 Al filed on Dec. 12, 2002.

Compounds disclosed in the above-cited Indian and U.S. patents and patent applications include for instance,

RS-(±)-9-fluoro-6,7-dihydro-8-(4-hydroxypiperidin-1-yl)-5-methyl-1-oxo-1H,5H-benzo[i,j]quinolizine-2-carboxylic acid, also referred herein as RS-(±)-nadifloxacin or nadifloxacin,

S-(−)-9-fluoro-6,7-dihydro-8-(4-hydroxypiperidin-1-yl)-5-methyl-1-oxo-1H,5H-benzo[i,j]quinolizine-2-carboxylic acid also referred to herein as S-(−)-nadifloxacin and hydrates thereof, and

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, also referred to herein as S-(−)-nadifloxacin arginine salt and polymorphs and hydrates thereof.

RS-nadifloxacin and S-nadifloxacin, in particular, exhibit strong antibacterial activity against Gram-positive, Gram-negative and anaerobic bacteria, resistant Gram-positive organisms such as methicillin-resistant Staphylococcus aureus (MRSA), quinolone-resistant Staphylococcus aureus, coagulase negative staphylococci, such as methicillin-resistant Staphylococcus epidermidis (MRSE), enterococci, betahemolytic streptococci and viridans group of streptococci, mycobacteria and newly emerging nosocomial pathogens such as Chryseobacterium meningosepticum, and Gram-negative pathogens such as E. coli, Klebsiella, Proteus, Serratia, Citrobacter and Pseudomonas. Recently, it has also been shown that S-(−)-nadifloxacin, in particular exhibits potent antibacterial activity against glycopeptide intermediate S. aureus (GISA), vancomycin intermediate S. aureus (VISA) and vancomycin-resistant S. aureus (VRSA).

Many Gram-positive organisms have developed significant levels of resistance to other antibiotics. About 65% of all cases of bacterial keratitis and about 85% of all cases of bacterial conjunctivitis are attributable to infection by gram-positive organisms such as those listed above. The causative organism of acne vulgaris is Propionibacterium acnes. The incidence of acne vulgaris is very high, specially among adolescents. Among new emerging diseases are pyoderma gangrenosum and necrotising fascitis. Pyoderma gangrenosum is a chronic destructive ulcerating wound disorder of unknown etiology, and pathophysiology. S. aureus is most often the infecting microorganism. Necrotising fascitis is a life threatening bacterial infection causing necrosis of the fascia, underlying skin and vasculature. It progresses rapidly, has a frightening 74% mortality and a high risk of systemic activity. The Group A beta-hemolytic streptococci (GABHS) are frequently identified in necrotising fascitis. Gonzalez M., Necrotising fascitis and gangrene of the upper extremity. Hand Clinics, 14(4), 635-645 (1998); Misago N. et.al., Necrotising fascitis due to group A Streptococci: A clinicopathology study of six patients. J. of Dermatology, 23, 876-883(1996). Another type of nacrotising fascitis involves several bacteria occurring with facultative and anaerobic bacteria. However, the infection is commonly polymicrobial in etiology (Geeham D M & Pemberton L B, Management of Wound Infection in the I.C.U. Critical Care Nursing Quarterly, 69-77, 1997, November), other causative enzymes including Clostridium, Peptococcus E. coli Pseudomonas, S. pyogenes S. aureus, S. epidermidis, S. marcescens (Douglas M, Necrotising fascitis: A nursing perspective, J. of Advanced Nursing, 24, 161-66, (1996);Gillen P B, Necrotising fascitis: Early recognition and aggressive treatment remain important, J. of Wound and Ostomy Care Nursing, 22 (5), 219-221, 1995). Bacterial resistance to antibiotics is an increasingly recurrent phenomenon. The need for pharmaceutical compositions specifically to treat infections arising from such emerging and resistant microorganisms thus assumes urgent significance.

The etiology of acne, its epidemiology, its psychosocial effects leading to impaired academic and social functioning, its effects on employment status, its consequences on the overall well-being and quality of life of the patient with acne, and the aims of treating it are described in U.S. Pat. Nos. 5,543,417 and 6,365,623 B1 and references contained therein, all of which are included herein by reference. Acne is a disease with multifactorial pathogenesis including among other factors that of proliferation of Propionibacterium acnes. Among conventional topical treatments such as the antibiotics erythromycin and esters thereof, neomycin, clindamycin and esters thereof, tetracycline or the more recent RS-(±)-9-fluoro-6,7-dihydro-8-(4-hydroxypiperidin-1-yl)-5-methyl-1-oxo-1H,5H-benzo[i,j]quinolizine-2-carboxylic acid, and anti-seborrhoeic or keratolytic agents such as benzoyl peroxide, salicylic acid, azelaic acid used for the removal of comedones in acne, are the topical retinoids such as tretinoin, isotretinoin and those listed in U.S. Pat. No. 4,717,720, U.S. Pat. No. 5,587,367 and U.S. Pat. No. 6,462,064 and references contained therein, all of which are included herein by reference. U.S. Pat. No. 5,587,367 discloses a pharmaceutical or cosmetic dermal composition containing a combination of a retinoid with a second agent, such a second agent being a sterol.

The compositions of this invention comprise a combination of an antibacterial benzoquinolizine-2-carboxylic acid such as herein described and a retinoid such as herein described resulting in a synergistic effect for the treatment of epidermic keratinization disorders, epithelial or epidermic proliferation disorders and/or disorders of the sebaceous function, for instance disorders selected from the group consisting of acne vulgaris, comedonic or polymorphic acne, acne rosaria, nodulocystic acne, acne conglobata, senile acne and secondary acnes.

Allergic inflammatory conditions of the skin are manifested by macules, papules or raised wheals involving part/s of the body. At cellular level there is a breakdown of phospholipids in the cell membrane and this gives rise to mediators like leukotrienes, platelet activating factor, prostaglandins and histamine. A steroid is generally administered to alleviate the symptoms of erythema, the immune response and the related itching which are normally associated with the above-mentioned group of bacterially-infected or invaded immunologic and/or allergic inflammatory dermal disorders. It is undesirable to use steroids alone for topical treatment for extended periods of time. Steroids can penetrate the skin and cause undesirable effects, including skin atrophy, suppression of the hypothalamic-pituitary-adrenal axis, Cushing's syndrome, glycosuria, hyperglycemia, etc. Combinations of antibacterials and steroids are disclosed in U.S. Pat. No. 4,604,384, WO 2002/039993, WO 02/30395 A1, WO 00/18404, U.S. Pat. No. 6,395,746, and WO 00/18404.

Fungal diseases refer to fungal infections, including yeast infections, of keratinized and non-keratinized epithelial tissues, for example skin, nails, mucosa and the like and includes tinea pedis, tinea capitis, tinea corporis, tinea versicolor, nail fungal diseases (distal subungual onychomycosis caused by dermatophyte infection), scalp disorders, tinea cruris, and candidiasis (cf. U.S. Pat. No. 6,075,056, incorporated herein by reference). Antifungal agents are useful in treating dermatophytoses such as trichophytid, endodermophytosis, favid and deepseated trichophytid and fungal infections such as mucocutaneous mycosis and deep-seated candidiasis (cf. WO 2000062776, incorporated herein by reference).

None of the references cited above specifically contemplates formulating a benzoquinolizine-2-carboxylic acid antibiotic in topical combination compositions using one or more ingredients selected from the group of a retinoid, an antibacterial, a steroid/non-steroid antiinflammatory agent and/or an antifungal agent.

None of the references cited above specifically contemplates formulating a benzoquinolizine-2-carboxylic acid antibiotic in a combination therapy or coformulation of a benzoquinolizine-2-carboxylic acid antibacterial agent having a high degree of activity against gram-positive bacterial with one or more antibacterial agents effective against gram-negative bacteria and/or with a retinoid, steroid/non-steroid anti-inflammatory agent and/or antifungal agent.

### SUMMARY OF THE INVENTION

It is an aspect of this invention to provide topical compositions of an antibacterial benzoquinolizine-2-carboxylic acid, incorporated either as the single therapeutic ingredient in pharmaceutical compositions, or as an ingredient in combination with at least one agent selected from the group of a retinoid, an antifungal agent, an antibacterial and/or a steroid/non-steroid anti-inflammatory agent, to processes for preparation of the compositions, to use of the compositions in preparation of a medicament, and to a method of therapeutic or prophylactic use of such a composition for the treatment of dermal, ophthalmic, otic and nasal infections, with or without attendant inflammation.

### DETAILED DESCRIPTION OF THE INVENTION

In accordance with this invention, any benzoquinolizine-2-carboxylic acid, antimicrobial drug or one of its chiral isomers i.e. one having a benzoquinolizine-2-carboxylic acid moiety as part of its chemical structure, can be formulated in a composition either as a single ingredient or in combination with one or more ingredients selected from the group of retinoid, an antifungal agent, an antibacterial and/or a steroid/non-steroid anti-inflammatory agent and one or more acceptable excipients, carriers, or diluents.

One embodiment of this invention relates to antibacterial benzoquinolizine-2-carboxylic acid-containing dermal compositions with at least one adjunct retinoid ingredient resulting in a synergistic effect for the treatment of epidermic keratinization disorders, epithelial or epidermic proliferation disorders and/or disorders of the sebaceous function, for instance disorders selected from the group consisting of acne vulgaris, comedonic or polymorphic acne, acne rosaria, nodulocystic acne, acne conglobata, senile acne and secondary acnes.

Another embodiment of this invention relates to antibacterial benzoquinolizine-2-carboxylic acid-containing dermal compositions with at least one steroid ingredient resulting in a synergistic effect for the treatment of bacterially infected or invaded immunologic and/or allergic inflammatory disorders, for instance selected from the group consisting of contact dermatitis, seborrhoeic dermatitis, erythema multiformae, pyodermic-related wounds and infective eczema and ophthalmic, otic or nasal disorders. The formulation of this invention has the advantages of combining an agent useful for treating the dermal and other body part/s bacterial diseases and disorders with a steroid capable of reducing the associated inflammation, with the ability to rapidly eradicate bacterial infections and eliminate the symptoms thereof, and as a consequence minimize the risk of undesirable side effects. Such a formulation would ideally deliver the antibacterial agent and the steroid to the skin and other body part/s, and maintain the combination on the skin and other body part/s for the period of time necessary to effect treatment, but minimize the penetration of the skin or other body part/s with respect to the active ingredients, thus avoiding the potential steroid effects noted above.

Still another embodiment of this invention relates to antibacterial benzoquinolizine-2-carboxylic acid-containing dermal and other body part/s compositions with at least one antifungal agent ingredient resulting in a synergistic effect for the treatment of bacterially infected fungal diseases.

An antifungal agent is any agent that prevents the growth of or kills a fungal organism such as antifungal polyene macrolides such as amphotericin B, and nystatin, azole antifungal agents such as clotrimazole, miconazole, and ketoconazole, arylmethylamine antifungal agents such as butenafine and terbinafine (cf. EP 0310122B1, incorporated herein by reference), fluorinated pyrimidines, halogenated phenolic ethers, thiocarbamates, allylamines, benzylamines. In addition, antifungal agents can be agents that interpolate fungal cell wall components or act as cell wall inhibitors. Specific antifungal agents within the scope of the invention include, without limitation, the squalene epoxidase inhibitor, butenafine, and the ergosterol biosynthesis inhibitor, miconazole.

Still another embodiment of this invention relates to an antibacterial benzoquinolizine-2-carboxylic acid-containing dermal and other body part/s compositions with at least one antifungal agent ingredient and at least one steroid resulting in a synergistic effect for the treatment of bacterially infected, inflammatory fungal diseases. WO 99/20261 (incorporated herein by reference) describes inflammation of mucosal tissue, fungus-induced mucositis and rhinositis, other fungus-induced mucositis conditions such as chronic otitis media, and methods and materials for treating them. Topical compositions are described for psoriatic infections (WO 9949835, incorporated herein by reference), and for cutaneous mycosis including candidiasis, vulvitis, etc., (JP 07233088 (incorporated herein by reference).

The subject antibiotic benzoquinolizine-2-carboxylic acid compounds, including but not limited to RS-(±)-9-fluoro-6,7-dihydro-8-(4-hydroxypiperidin-1-yl)-5-methyl-1-oxo-1H,5H-benzo[i,j]quinolizine-2-carboxylic acid, S-nadifloxacin, S-nadifloxacin arginine salt, can be formulated as a gel or cream for topical application to skin, or they can be formulated as an ointment or gel; or eye drops for application to a mammalian eye.

Preferred benzoquinolizine-2-carboxylic acid are compounds having Formula-I

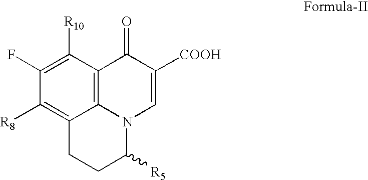

Preferably R₅ is C₁₋₆ alkyl, and more preferably R₅ is CH₃, as a mixture of enantiomers or in a stereochemical orientation.

Preferably R₈ is 4-hydroxypiperidinyl optionally further substituted with one or more C₁₋₆ alkyl, hydroxypiperidinyl optionally further mono/poly substituted with C₁₋₆ alkyl.

More preferably R₈ is

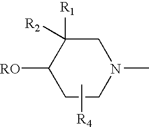

wherein

R is hydrogen, C₁-C₆ alkyl, glycosyl, or aralkyl such as benzyl, or R is C₁-C₆ alkanoyl such as acetyl, propionyl, or pivaloyl; or aminoalkanoyl such as amino acid residues derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof, or R is C₆H₁₁O₆, PO₃H₂ or SO₃H thus giving respectively the gluconic acid, phosphoric acid and sulfonic acid ester derivatives of the compounds;

R₁ and R₂ are the same or different and represent H, C₁₋₄ alkyl, aralkyl, aminoalkyl, trifluoroalkyl, or halogen;

R₄ is selected from H, C₁₋₄ alkyl, CF₃, phenyl, or F and R₄ is present at one or more of the positions of 2-, 4-, 5-, or 6- of the piperidine ring;

R₁₀ is H, C₁₋₅ alkyl, amino, alkylamino, or acylamino;

or an optical isomer, diastereomer or enantiomer thereof, or a polymorph, pseudopolymorph, or prodrug thereof or a pharmaceutically acceptable salt or hydrate thereof.

“Optical isomer”, “stereoisomer”, and “diastereomer” as referred to herein have the standard art recognized meanings.

Examples of preferred benzoquinolizine-2-carboxylic acid are compounds selected from RS-(±)-, R-(+)- or S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and polymorphic forms thereof, RS-(±)-, R-(+)- or S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate, RS-(±)-, R-(+)- or S-(−)-9-fluoro-6,7-dihydro-8-(4-hydroxypiperidin-1-yl)-5-methyl-1-oxo-1H,5H-benzo[i,j]quinolizine-2-carboxylic acid, S-(−)-9-fluoro-6,7-dihydro-8-{trans-4-(RS)-hydroxy-3-(RS)-methylpiperidin-1-yl}-5-methyl-1-oxo-1H,5H-benzo[i,j]quinolizine-2-carboxylic acid, S-(−)-9-fluoro-6,7-dihydro-8-{cis-4-(RS)-hydroxy-3-(RS)-methylpiperidin-1-yl-5-methyl- -oxo-1H,5H-benzo[i,j]quinolizine-2-carboxylic acid, S-(−)-9-fluoro-6,7-dihydro-8-{cis-(−)4-R-hydroxy-3-S-methylpiperidin-1-yl}-5-methyl-1-oxo-1H,5H-benzo[i,j]quinolizine-2-carboxylic acid, S-(−)-9-fluoro-6,7-dihydro-8-{cis-(+)-4-S-hydroxy-3-R-methylpiperidin-1-yl}-5-methyl-l -oxo-1H,5H-benzo[i,j]quinolizine-2-carboxylic acid, S-(−)-9-fluoro-6,7-dihydro-8-(3-ethyl-4-hydroxypiperidin-1-yl)-5-methyl-1-oxo-1H,5H-benzo[i,j]quinolizine-2-carboxylic acid (mixture of cis racemate and trans racemate) and pure stereoisomers thereof, RS-(±)-9-fluoro-6,7-dihydro-8-(4-hydroxypiperidin-1-yl)-5-methyl-1-oxo-1H,5H-benzo[i,j]quinolizine-2-carboxylic acid, also referred herein as RS-(±)-nadifloxacin,

S-(−)-9-fluoro-6,7-dihydro-8-(4-hydroxypiperidin-1-yl)-5-methyl-1-oxo-1H,5H-benzo[i,j]quinolizine-2-carboxylic acid also referred to herein as S-(−)-nadifloxacin, and

S-(−)-9-fluoro-6,7-dihydro-8-(4-hydroxypiperidin-1-yl)-5-methyl-1-oxo-1H,5H-benzo[i,j]quinolizine-2-carboxylic acid arginine salt, also referred to herein as S-(−)-nadifloxacin arginine salt.

Benzoquinolizine compounds used in compositions of the invention can be prepared by a process known per se, by processes described in the patents included herein by reference disclosing such drugs.

U.S. Pat. No. 4,399,134

U.S. Pat. No. 4,552,879

U.S. Pat. No. 6,514,986;

U.S. Pat. No. 6,608,669;

U.S. application Ser. No. 09/566,875

U.S. application Ser. No. 09/640,947

U.S. application Ser. No. 10/156,685

Antibiotics that can be used in combination with an antibacterial benzoquinolizine-2-carboxylic acid compound may include but are not limited to: Polymyxin Sulphate(Gram-ve), Neomycin, Bacitracin, Trimethoprin, Tobramycin, Terramycin, Sulfacetamide, Cefazolin, Gramicidin and Colistin Sulphate(Gram-ve).

Retinoids, antiacne agents, steroids (glucocorticoids) and antifungal agents that can be used in the compositions of this invention include but are not limited to:

Retinoids: Benzoyl peroxide, Dichloroacetic acid, Glutaraldehyde, Resorcinol, Retinoic acid and Salicylic acid.

Antiacne: Adapalene, Algestone acetophenide, Azelaic acid, Benzoyl peroxide, Cioteronel, Cyproterone, Isotretinoin, Motretinide, Resorcinol, Retinoic acid, Tretinoin, Tazarotene and Tioxolone.

Glucocorticoid: 21-acetoxypregnnolone, Alclometasone, Algestone, Amcinonide, Beclomethasone, Betamethasone, Budesonide, Chloroprednisone, Ciclesonide, Clobetasol, Clobetasone, Clocortolone, Cloprednol, Corticosterone, Cortisone, Cortivazol, Deflazacort, Desonide, Desoximetasone, Dexamethasone, Diflorasone, Diflucortolone, Difluprednate, Enoxolone, Fluazacort, Flucloronide, Flumethasone, Flunisolide, Fluocinolone acetonide, Fluocinonide, Fluocortin butyl, Fluocortolone, Fluorometholone, Fluperolone acetate, Fluprednidene acetate, Fluprednidene acetate, Fluprednisolone, Flurandrenolide, Fluticasone propionate, Formocortal, Halcinonide, Halobetasol propionate, Halometasone, Halopredone acetate, Hydrocortamate, Hydrocortisone, Loteprednol etabonate,Mazipredone, Medrysone, Meprednisone, Methylprednisolone, Mometasone furoate, Paramethasone, Prednicarbate, Prednisolone, Prednisolone 21-diethylaminoacetate, Prednisolone sodium phosphate, Predisone, prednival, Prednylidene, Rimexolone, Tixocortol, Triamcinolone, Triamcinoloneacetonide, Triamcinolone benetonide, Triamcinolone hexacetonide.

Antifungal (Antibiotics): Polyenes: Amphotericin, Candicidin, Dermostatin, Filipin, Fungichromin, Hachimycin, Hamycin, Lucensomycin, Mepartricin, Natamycin, Nystatin, Pecilocin, Perimycin, Azaserine, Caspofungin, Griseofulvin, Oligomycins, Pyrrolnitrin, Siccanin, Tubercidin, Viridin.

Antifungal (Synthetic): Allylamines: Butenafine, Naftifine, Terbinafine Imidazoles: Bifonazole, Butoconazole, Chlordantoin, Chlormidazole, Cloconazole, Clotrimazole, Econazole, Enilconazole, Fenticonazole, Flutrimazole, Isoconazole, Ketoconazole, Lanoconazole, Miconazole, Neticonazole, Omoconazole, Oxiconazole nitrate, Sertaconazole, Sulconazole, Tioconazole.

Thiocarbamates: Liranaftate, Tolciclate, Tolindate, Tolnaftate Triazoles: Fluconazole, Itraconazole, Posaconazole, Saperconazole, Terconazole, Voriconazole.

Others: Acrisorcin, Amorolfine, Biphenamine, Bromosalicylchloranilide, Buclosamide, Calcium propionate, Chlorphenesin, Ciclopirox, Cloxyquin, Coparaffinate, Diamthazole dihydrochloride, Exalamide, Flucytosine, Hexetidine, Loflucarban, Nedocromil sodium, Nifuratel, Potassium iodide, Propionic acid, Pyrithione, Salicylanilide, Sodium propionate, Sulbentine, Tenonitrozole, Triacetin, Undecylenic acid, Zinc propionate

The preferred retinoid is adapalene.

The preferred steroid is clobetasol or mometasone, in particular, clobetasol propionate.

The preferred antifungal agent is butenafine.

The compositions of this invention contain from about 0.1 to 10% by weight of the composition of an antimicrobial benzoquinolizine-2-carboxylic acid compound.

Preferably the amount of the antimicrobial benzoquinolizine-2-carboxylic acid compound in the composition is 1% by weight of the total weight of the composition. More preferably, the amount of the antimicrobial benzoquinolizine-2-carboxylic acid compound in the composition is 0.5% by weight of the total weight of the composition.

Preferably, the amount of the retinoid present in a composition of this invention is 0.001 to 10.0% by weight relative to the total weight of the composition. Preferably, the amount of the steroid present in a composition of this invention is 0.005-1.0% by weight relative to the total weight of the composition.

Preferably, the amount of the antifungal agent present in a composition of this invention is 0.1 to 10.0% by weight relative to the total weight of the composition.

The compositions of this invention may be in a physical form selected from concentrates, drops, pastes, ointments, creams, milks, pomades, powders, impregnated pads, tulles, solutions, gels, sprays, shampoos, lotions, suspensions, microspheres, nanospheres, lipidic vesicles, polymeric vesicles, polymeric patches or biological inserts.

The route of administration of the compositions is selected from ocular, nasal, otic, rectal, vaginal, intradermal, intratumoral, intralesional, intravascular, topical, transdermal, local, regional, or loco-regional.

In addition to the benzoquinolizine-2-carboxylic acid and/or one of its combination partners mentioned above, the compositions will also include a pharmaceutical vehicle compatible with an administration by a topical method (skin and mucous), ocular, otic or nasal or the other routes of administration described herein.

For topical application, the pharmaceutical or cosmetic compositions of the invention comprise the vehicles and ingredients required to provide the composition, for example, in the form of ointments, creams, milks, pomades, powders, impregnated pads, solutions, gels, sprays, shampoos, washing lotions or even suspensions, microspheres or nanospheres, lipidic or polymeric vesicles or polymeric patches.

For ocular administration, the composition of the invention is provided in the form of eyedrops or eyewashes.

In those embodiments of the present invention wherein the pharmaceutical composition is in the form of ointments, creams, lotions, gels and the like for dermal application, the vehicle may also contain other pharmaceutically acceptable excipients known in the art for pharmaceutical compounding such as for example, penetration enhancers, humectants and/or moisturizers, preservatives, opacifiers, fragrances, color additives, counter-irritants and the like.

The penetration enhancers, for improved transepidermal or percutaneous delivery of drug, suitable for the present invention include terpenes, terpene alcohols, essential oils, surfactants, and the like. Some such examples include d-limonene, terpinen-4-ol, menthone, 1,8-cineole, 1-pinene, α-terpineol, carveol, carvone, pulegone, eucalyptol, peppermint oil, sorbitan esters, polysorbates, sodium lauryl sulphate, and the like.

Suitable humectants and/or moisturizers that may be used in the present invention include polyhydroxy alcohols such as sorbitol, glycerin, hexanetriol, butanediol, mannitol, glucose, ethylene glycol, propylene glycol, and the like.

Preservatives such as methylparaben, propylparaben, phenoxyethanol, benzyl alcohol, bromopol, chlorocresol, thiomersal, benzalkonium chloride, and the like may be added to the compositions to inhibit microbial activity.

Opacifiers, such as behenic acid, glycol distearate, lard glycerides, polyethylene glycol esters, and the like; fragrances such as amyl salicylate, panisaldehyde, anisylalcohol, peppermint oil, wintergreen oil, and the like; colour additives such as quinoline yellow, and the like; counter-irritants such as methyl salicylate, menthol and the like; and other pharmaceutical adjuvants may be added to the compositions of the invention.

Antiforming agents such as simethicon and dimethicon may be added to the compositions.

In those embodiments of the present invention wherein the pharmaceutical composition is in the form of solutions, suspensions, ointments, gels and the like for ocular administration, the vehicle may also contain other ophthalmically acceptable excipients known in the art for pharmaceutical compounding such as for example, solvents, fillers, buffering agents, tonicity regulators, viscosity enhancers, lubricity components, chelating/sequestering agents, stabilizing agents, and the like.

As used herein, the term “ophthalmically acceptable” refers to an excipient which, at the concentration or amount in question, is compatible with ocular tissue and does not cause significant or undue detrimental effects when brought into contact with ocular tissue.

In those embodiments of the present invention wherein the pharmaceutical composition is intended for ocular administration, it may contain water, mixtures of water and water-miscible solvents such as lower alkanols or aralkanols, vegetable oils, polyalkylene glycols, petroleum-based jelly, ethyl oleate, isopropyl myristate, and the like as solvents; polyethylene glycols, carbowaxes, petroleum jelly and the like as a fillers; tromethamine, phosphate, borate, acetate, citrate buffers, and the like as a buffering agents; dextrose, potassium chloride, sodium chloride, and the like as a tonicity regulators; carbopol, ethyl cellulose, hydroxypropyl methylcellulose, sodium carboxymethyl cellulose, carboxymethyl cellulose, hydroxyethyl cellulose and the like as a viscosity enhancers; polyvinyl alcohol, polyvinylpyrrolidone, carbopol and the like as a lubricity components; ethylene diamine tetraacetic acid (EDTA), citric acid, tartaric acid, and the like as a chelating or sequestering agents; and antioxidants, for example, alkali metal metabisulfates, ascorbic acid, and the like as a stabilizing agent.

In an embodiment of the process of the present invention, the present compositions may be prepared using conventional techniques, for example, by formation of solutions, gels, suspensions, etc., using well known and conventional techniques. For a more detailed discussion of the preparation and administration of ophthalmic formulations see Remington's Pharmaceutical Sciences, 15 Ed., Pgs. 1489 to 1504 (1975) which is incorporated in its entirety herein by reference. Compositions of the present invention can also be prepared by processes known in the art, including by simple admixture, with agitation as appropriate, of the ingredients. The processes for preparing a composition of the invention are preferably conducted so as to provide a sterile product. According to an embodiment of the present invention, the container into which the dosage forms are dispensed could be made of glass, plastic, aluminum, and the like. In this connection, the container materials can contain substances that confer a particular protection on the contents, such as, for example, a protection from light or a protection from oxygen.

The invention also has for an object the use of the ingredients of the invention in the preparation of a pharmaceutical or cosmetic composition intended principally for the treatment or correction of epidermic keratinization disorders, any other disorder or any other functional defect or excess of epidermic or epithelial proliferation. The composition thus prepared can serve to treat the disorders mentioned above, having or not an inflammatory and/or immunoallergic component, comprising conjunctive tissue degeneration disorders and benign or malignant tumors, to combat against skin aging, to favor cicatrization or to improve the appearance of the skin of persons exhibiting keratinization disorders or suffering from seborrhea. The invention also has for an object the use of the ingredients of the invention in the preparation of a pharmaceutical composition intended for the treatment of ocular and periocular infections.

In particular, the combination described in the present invention is intended:

for the treatment of dermatologic ailments linked to a keratinization disorder causing differentiation and proliferation and principally for treating common acne, comedons, polymorphs, nodulokystic acne, conglobuta, senile acne, secondary acne such as solar, medicinal and professional acne;

for the treatment of other types of keratinization disorders, principally ichthyoses, ichthyosiform conditions, Darier malady, palmoplantary keratodermies, leucophasies and leucoplasiform conditions as well as lichen;

for the treatment of dermatologic ailments linked to a keratinization disorder having an inflammatory and/or immunoallergic component and principally, all forms of psoriasis, be they cutaneous, mucous or ungual, and even psoriasic rheumatism, or again cutaneous atopies, such as eczema;

for the treatment of other dermatologic disorders such as blistery dermatoses and collagen maladies;

to prevent or heal scars of epidermic and/or dermic atrophy, induced by local or systemic corticosteroids, or any other form of cutaneous atrophy;

for the treatment of certain ophthalmologic disorders, principally corneopathies; bacterial keratitis, corneal ulcers, bacterial conjunctivitis, associated or unassociated with allergy or itch to combat against disorders of the sebaceous function such as hyperseborrhea of acne or simple seborrhea;

to combat impetigo folliculitis, infected dermatitis, wounds and burns, pyoderma gangrenosum and necrotising fascitis.

The compositions of the invention are also useful in the cosmetic field, in particular in body hygiene, and also capillary hygiene (action against seborrhea).

Generally the composition for topical use is applied one or more times per day on the area to be treated. The number of times a time per day that the composition is applied depends on the severity of the condition and the advice of the physician.

In general, the present methods for treating mammalian eyes comprise administering to the mammalian eye a therapeutically effective amount of the present composition thereby providing an effective antibiotic in the mammalian eye, and, if a combination partner component is present in the composition, thereby reducing inflammation or pain in the mammalian eye. The present methods of use may involve any suitable administration step or steps to provide an effective amount of the composition to the mammalian eye. Such administering may include, but is not limited to, topical application to the eye, instillation into the eye, placing an insert into the cul-de-sac (space) between the eyeball and the eyelid and the like. Other conventional methods of administering compositions to the eye may be employed provided that the present compositions are administered so as to provide the benefits desired.

The present use methods may be considered to be curative and/or preventative when applied, presurgically or immediately post traumatically, that is before a microbial infection develops, or before inflammation and/or pain is apparent. The present use methods are effective to reduce the risk of the formation of such infections and to reduce the severity of any inflammation or pain which may develop.

The dosage level of the present composition depends, of course, on many factors, for example, the particular application involved, the particular active component or components employed, the concentration of the active component or components in the composition, the severity of the infection/inflammation/pain and the individuals response to the treatment. Such dosage can be easily determined by routine and well known techniques to achieve the desired results in the individual patient being treated.

The following non-limiting examples illustrate certain aspects of the present invention.

### EXAMPLE 1

This example illustrates the present invention in the form of a gel of RS-(±)-9-fluoro-6,7-dihydro-8-(4-hydroxypiperidin-1-yl)-5-methyl-1-oxo-1H,5H benzo[i,j]quinolizine-2-carboxylic acid. The pharmaceutical composition of this example is given below in Table 1;

TABLE 1

| Ingredients | % weight of the Composition |
|---|---|
| Active antibacterial | 1.00 |
| Carbopol | 1.20 |
| Sodium hydroxide | 0.112 |
| Diethanolamine | 0.36 |
| Disodium edetate | 0.10 |
| Sodium sulfite anhydrous | 0.05 |
| Purified water | q.s. to 100.00 |

Sodium hydroxide, diethanolamine, sodium sulfite anhydrous and the active antibacterial ingredient were dissolved in purified water (Solution A). Carbopol was dispersed in 50% w/v aqueous solution of disodium edetate to which was added Solution A and mixed to form a gel.

### EXAMPLE 2

This example illustrates the present invention in the form of a cream of a combination of RS-(±)-9-fluoro-6,7-dihydro-8-(4-hydroxypiperidin-1-yl)-5-methyl-1-oxo-1H,5H benzo[i,j]quinolizine-2-carboxylic acid and adapalene. The pharmaceutical composition of this example is given below in table 2.

TABLE 2

| Ingredients | % Weight of the composition |
|---|---|
| Active antibacterial | 1.00 |
| Adapalene | 0.10 |
| Disodium edetate | 0.10 |
| Propylene glycol | 10.00 |
| Methyl paraben | 0.18 |
| Propyl paraben | 0.02 |
| Cetostearyl alcohol | 7.20 |
| Liquid paraffin (heavy) | 15.00 |
| Microcrystalline wax | 3.00 |
| Cetomacrogol - 1000 | 2.00 |
| Dimethicone | 0.10 |
| α-Tocopherol | 0.03 |
| Purified water | q.s. to 100.00 |

Cetostearyl alcohol, liquid paraffin, microcrystalline wax, cetomacrogol and dimethicone were mixed and heated to 70° C. An aqueous solution of disodium edetate was also heated to 70° C. and added to the above mixture under homogenization to form an emulsion. Dispersion of methyl paraben and propyl paraben in propylene glycol was added to the emulsion, which was cooled to room temperature to form a cream. α-tocopherol was further dispersed in this cream following which the active antibacterial ingredient and adapalene were triturated with part quantity of cream and then mixed with entire quantity of cream. The cream was passed through triple roller mill prior to filling in the tubes.

### EXAMPLE 3

This example illustrates the present invention in the form of a gel of a combination of RS-(±)-9-fluoro-6,7-dihydro-8-(4-hydroxypiperidin-1-yl)-5-methyl-1-oxo-1H,5H benzo[i,j]quinolizine-2-carboxylic acid and adapalene. The pharmaceutical composition of this example is given below in table 3.

TABLE 3

| Ingredients | % weight of the Composition |
|---|---|
| Active antibacterial | 1.00 |
| Adapalene | 0.10 |
| Carbopol | 1.20 |
| Sodium hydroxide | 0.112 |
| Diethanolamine | 0.36 |
| Disodium edetate | 0.10 |
| Sodium sulfite anhydrous | 0.05 |
| N-methyl-2-pyrrolidone | 2.50 |
| Methyl paraben | 0.18 |
| Propyl paraben | 0.02 |
| Propylene glycol | 5.00 |
| Purified water | q.s. to 100.00 |

Sodium hydroxide, diethanolamine, sodium sulfite anhydrous and RS-(±)-9-fluoro-6,7-dihydro-8-(4-hydroxypiperidin-1-yl)-5-methyl-1-oxo-1H,5H-benzo[i,j]quinolizine-2-carboxylic acid were dissolved in purified water (Solution A). Adapalene was dissolved in n-methyl-2-pyrrolidone (Solution B). Methyl paraben and propyl paraben were dissolved in propylene glycol (Solution C). In a 50% (w/v) aqueous solution of disodium edetate were added all the solutions A, B and C, one by one, and mixed to form a gel.

### EXAMPLE 4

This example illustrates the present invention in the form of a cream of a combination of RS-(±)-9-fluoro-6,7-dihydro-8-(4-hydroxypiperidin-1-yl)-5-methyl-1-oxo-1H,5H benzo[i,j]quinolizine-2-carboxylic acid and a steroid, clobetasol, propionate. The pharmaceutical composition of this example is given below in table 4.

TABLE 4

| Ingredients | % Weight of the composition |
|---|---|
| Active antibacterial | 1.00 |
| Clobetasol Propionate | 0.05 |
| Disodium edetate | 0.10 |
| Propylene glycol | 10.00 |
| Methyl paraben | 0.18 |
| Propyl paraben | 0.02 |
| Cetostearyl alcohol | 7.20 |
| Liquid paraffin (heavy) | 15.00 |
| Microcrystalline wax | 3.00 |
| Cetomacrogol - 1000 | 2.00 |
| Dimethicone | 0.10 |
| α-Tocopherol | 0.03 |
| Purified water | q.s. to 100.00 |

Cetostearyl alcohol, liquid paraffin, microcrystalline wax, cetomacrogol and dimethicone were mixed and heated to 70° C. An aqueous solution 40% (w/v) of disodium edetate was also heated to 70° C. and added to the above mixture under homogenization to form an emulsion. Dispersion of methyl paraben and propyl paraben in a part of propylene glycol was added to the emulsion, which was cooled to room temperature to form a cream. α-tocopherol was further dispersed in this cream. Clobetasol propionate dissolved in the remaining portion of propylene glycol, and the active antibacterial ingredient were triturated with part quantity of cream and then mixed with the entire quantity of cream. The cream was passed through triple roller mill prior to filling in the tubes.

### EXAMPLE 5

This example illustrates the present invention in the form of a gel of a combination of RS-(±)-9-fluoro-6,7-dihydro-8-(4-hydroxypiperidin-1-yl)-5-methyl-1-oxo-1H,5H benzo[i,j]quinolizine-2-carboxylic acid and clobetasol propionate. The pharmaceutical composition of this example is given in below in table 5.

TABLE 5

| Ingredients | % weight of the Composition |
|---|---|
| Active antibacterial | 1.00 |
| Clobetasol propionate | 0.05 |
| Carbopol | 1.20 |
| Sodium hydroxide | 0.112 |
| Diethanolamine | 0.36 |
| Disodium edetate | 0.10 |
| Propylene glycol | 15.00 |
| Sodium sulfite anhydrous | 0.05 |
| Methyl paraben | 0.18 |
| Propyl paraben | 0.02 |
| Purified water | q.s. to 100.00 |

Sodium hydroxide, diethanolamine, sodium sulfite anhydrous and the active antibacterial ingredient were dissolved in purified water (Solution A). Paraben was dissolved in propylene glycol (Solution B). Clobetasol propionate was dissolved in propylene glycol (Solution C). Carbopol was dispersed in an aqueous solution 50% (w/v) of disodium edetate to which was added Solutions A, B and C, one by one, and mixed to form a gel.

### EXAMPLE 6

This example illustrates the present invention in the form of a cream of a combination of RS-(±)-9-fluoro-6,7-dihydro-8-(4-hydroxypiperidin-1-yl)-5-methyl-1-oxo-1H,5H benzo[i,j]quinolizine-2-carboxylic acid, clobetasol propionate and butenafine hydrochloride. The pharmaceutical composition of this example is given below in table 6.

TABLE 6

| Ingredients | % Weight of the composition |
|---|---|
| Active antibacterial | 1.00 |
| Butenafine Hydrochloride | 1.00 |
| Clobetasol Propionate | 0.05 |
| Disodium edetate | 0.10 |
| Propylene glycol | 10.00 |
| Methyl paraben | 0.18 |
| Propyl paraben | 0.02 |
| Cetostearyl alcohol | 7.20 |
| Liquid paraffin (heavy) | 15.00 |
| Microcrystalline wax | 3.00 |
| Cetomacrogol - 1000 | 2.00 |
| Dimethicone | 0.10 |
| α-Tocopherol | 0.03 |
| Diethanolamine | 0.30 |
| Purified water | q.s. to 100.00 |

Cetostearyl alcohol, liquid paraffin, microcrystalline wax, cetomacrogol and dimethicone were mixed and heated to 70° C. An aqueous solution 45% (w/v) of disodium edetate was also heated to 70° C. and added to the above mixture under homogenization to form an emulsion. Dispersion of methyl paraben and propyl paraben in a part of propylene glycol was added to the emulsion, which was cooled to room temperature to form a cream. α-tocopherol was further dispersed in this cream. Clobetasol propionate dissolved in the remaining portion of propylene glycol, active antibacterial ingredient and butenafine hydrochloride were triturated with part quantity of cream and then mixed with entire quantity of cream. Diethanolamine was further mixed in and the cream was passed through triple roller mill prior to filling in the tubes.

### EXAMPLE 7

This example illustrates the present invention in the form of a cream of a combination of RS-(±)-9-fluoro-6,7-dihydro-8-(4-hydroxypiperidin-1-yl)-5-methyl-1-oxo-1H,5H benzo[i,j]quinolizine-2-carboxylic acid, clobetasol propionate and miconazole nitrate. The pharmaceutical composition of this example is given below in table 7.

TABLE 7

| Ingredients | % Weight of the composition |
|---|---|
| Active antibacterial | 1.00 |
| Miconazole Nitrate | 2.00 |
| Clobetasol Propionate | 0.05 |
| Disodium edetate | 0.10 |
| Propylene glycol | 10.00 |
| Methyl paraben | 0.18 |
| Propyl paraben | 0.02 |
| Cetostearyl alcohol | 7.20 |
| Liquid paraffin (heavy) | 15.00 |
| Microcrystalline wax | 3.00 |
| Cetomacrogol - 1000 | 2.00 |
| Dimethicone | 0.10 |
| α-Tocopherol | 0.03 |
| Diethanolamine | 0.30 |
| Purified water | q.s. to 100.00 |

Cetostearyl alcohol, liquid paraffin, microcrystalline wax, cetomacrogol and dimethicone were mixed and heated to 70° C. An aqueous solution 43% (w/v) of disodium edetate was also heated to 70° C. and added to the above mixture under homogenization to form an emulsion. Dispersion of methyl paraben and propyl paraben in a part of propylene glycol was added to the emulsion, which was cooled to room temperature to form a cream. α-tocopherol was further dispersed in this cream. Clobetasol propionate dissolved in the remaining portion of propylene glycol, active antibacterial ingredient and miconazole nitrate were triturated with part quantity of cream and then mixed with entire quantity of cream. Diethanolamine was further mixed in, and the cream was passed through triple roller mill prior to filling in the tubes.

### EXAMPLE 8

This example illustrates the present invention in the form of a cream of a combination of RS-(±)-9-fluoro-6,7-dihydro-8-(4-hydroxypiperidin-1-yl)-5-methyl-1-oxo-1H,5H benzo[i,j]quinolizine-2-carboxylic acid and miconazole nitrate. The pharmaceutical composition of this example is given below in table 8.

TABLE 8

| Ingredients | % Weight of the composition |
|---|---|
| Active antibacterial | 1.00 |
| Miconazole Nitrate | 2.00 |
| Disodium edetate | 0.10 |
| Propylene glycol | 10.00 |
| Methyl paraben | 0.18 |
| Propyl paraben | 0.02 |
| Cetostearyl alcohol | 7.20 |
| Liquid paraffin (heavy) | 15.00 |
| Microcrystalline wax | 3.00 |
| Cetomacrogol - 1000 | 2.00 |
| Dimethicone | 0.10 |
| α-Tocopherol | 0.03 |
| Diethanolamine | 0.30 |
| Purified water | q.s. to 100.00 |

Cetostearyl alcohol, liquid paraffin, microcrystalline wax, cetomacrogol and dimethicone were mixed and heated to 70° C. An aqueous solution 40% (w/v) of disodium edetate was also heated to 70° C. and added to the above mixture under homogenization to form an emulsion. Dissolved methyl paraben and propyl paraben in propylene glycol and added to the emulsion, which was cooled to room temperature to form a cream. α-tocopherol was further dispersed in this cream. Active antibacterial ingredient and miconazole nitrate were triturated with part quantity of cream and then mixed with entire quantity of cream. Diethanolamine was further mixed in, and the cream was passed through triple roller mill prior to filling in the tubes.

1. A stable pharmaceutical composition comprising:
a pharmaceutically effective amount of benzoquinolizine-2-carboxylic acid antimicrobial drug of the formula: 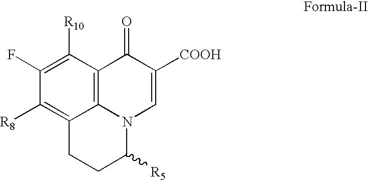
wherein:
R₅ is C₁₋₆ alkyl, as a mixture of enantiomers or in a stereochemical orientation;
R₈ is 4-hydroxypiperidinyl optionally further substituted with one or more C₁₋₆ alkyl, hydroxypiperidinyl optionally further mono/poly substituted with C₁₋₆ alkyl;
R₁₀ is selected from H, C₁₋₅ alkyl, amino, alkylamino and acylamino groups;
or an optical isomer, diastereomer or enantiomer thereof, or polymorphs and pseudopolymorphs or prodrugs thereof or pharmaceutically acceptable salts and hydrates thereof or mixtures thereof; singly or in combination with
a pharmaceutically effective amount(s) of a retinoid, an antibacterial, a steroid/non-steroid antiinflammatory agent, an antifungal agent or mixtures thereof:

2. The composition of claim 1 wherein in the formula (I),
R₅ is CH₃, in S-orientation.
R₈ is 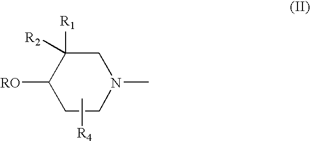
wherein:
R is hydrogen, C₁-C₆ alkyl, glycosyl, aralkyl, C₁-C₆ alkanoyl, or aminoalkanoyl or R is C₆H₁₁O₆, PO₃H₂ or SO₃H thus giving respectively the gluconic acid, phosphoric acid and sulfonic acid ester derivatives of the compounds;
R₁ and R₂ are the same or different and represent H, C₁₋₄ alkyl, aralkyl, aminoalkyl, trifluoroalkyl or halogen;
R₄ is H, C₁₋₄ alkyl, CF₃, phenyl, or F; R₄ is present at one or more of the positions of 2-, 4-, 5-, or 6- of the piperidine ring; and
R₁₀ is selected from H, C₁₋₅ alkyl, amino, alkylamino or acylamino groups.

3. The composition of claim 1 wherein the benzoquinolizine-2-carboxylic acid antimicrobial drug is selected from the group consisting of
RS-(±)-9-fluoro-6,7-dihydro-8-(4-hydroxypiperidin-1-yl)-5-methyl-1-oxo-1H,5H-benzo[i,j]quinolizine-2-carboxylic acid;
R(+)-9-fluoro-6,7-dihydro-8-(4-hydroxypiperidin-1-yl)-5-methyl-1-oxo-1H,5H-benzo[i,j]quinolizine-2-carboxylic acid;
S-(−)-9-fluoro-6,7-dihydro-8-(4-hydroxypiperidin-1-yl)-5-methyl-1-oxo-1H,5H-benzo[i,j]quinolizine-2-carboxylic acid;
RS-(±)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
R(+)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof;
RS-(±)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
R(+)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate;
S-(−)-9-fluoro-6,7-dihydro-8-{trans-4-(RS)-hydroxy-3-(RS)-methylpiperidin-1-yl}-5-methyl-1-oxo-1H,5H-benzo[i,j]quinolizine-2-carboxylic acid;
S-(−)-9-fluoro-6,7-dihydro-8-{cis-4-(RS)-hydroxy-3-(RS)-methylpiperidin-1-yl-5-methyl-oxo-1H,5H-benzo[i,j]quinolizine-2-carboxylic acid;
S-(−)-9-fluoro-6,7-dihydro-8-{cis-(−)-4-R-hydroxy-3-S-methylpiperidin-1-yl}-5-methyl-1-oxo-1H,5H-benzo[i,j]quinolizine-2-carboxylic acid;
S-(−)-9-fluoro-6,7-dihydro-8-{cis-(+)-4-S-hydroxy-3-R-methylpiperidin-1-yl}-5-methyl-1-oxo-1H,5H-benzo[i,j]quinolizine-2-carboxylic acid; and
S-(−)-9-fluoro-6,7-dihydro-8-(3-ethyl-4-hydroxypiperidin-1-yl)-5-methyl-1-oxo-1H,5H-benzo[i,j]quinolizine-2-carboxylic acid (mixture of cis racemate and trans racemate) and pure stereoisomers thereof.

4. The composition of claim 3, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof.

5. The composition of claim 3, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

6. The composition of claim 3, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid.

7. The composition of claim 1, wherein the benzoquinolizine-2-carboxylic acid antimicrobial drug comprises about 0.1-10% by weight of the composition.

8. The composition of claim 1 wherein the benzoquinolizine-2-carboxylic acid antimicrobial drug comprises about 1% by weight of the composition.

9. The method of claim 1 wherein the benzoquinolizine-2-carboxylic acid antimicrobial drug comprises about 0.5% by weight of the composition.

10. The composition of claim 1 wherein said retinoid is selected from the group consisting essentially of benzoyl peroxide, dichloroacetic acid, glutaraldehyde, resorcinol, retinoic acid, salicylic acid, adapalene, algestone acetophenide, azelaic acid, benzoyl peroxide, cioteronel, cyproterone, isotretinoin, motretinide, tretinoin, tazarotene, tioxolone, combinations and mixtures thereof.

11. The composition of claim 9, wherein the retinoid comprises adapalene.

12. The composition of claim 1, wherein said antibacterial is selected from the classes of aminoglycosides, cephalosporins, diaminopyridines, oxazolidinones, sulfonamides, tetracyclines or combinations of these classes.

13. The composition of claim 1, wherein said steroid is selected from the group consisting essentially of 21-acetoxypregnnolone, alclometasone, algestone, amcinonide, beclomethasone, betamethasone, budesonide, chloroprednisone, ciclesonide, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 21-diethylaminoacetate, prednisolone sodium phosphate, predisone, prednival, prednylidene, pimexolone, tixocortol, triamcinolone, triamcinoloneacetonide, triamcinolone benetonide, triamcinolone hexacetonide, combinations and mixtures thereof.

14. The composition of claim 13, wherein the steroid comprises clobetasol.

15. The composition of claim 13, wherein the steroid comprises mometasone.

16. The composition of claim 1, wherein said non-steroid antiinflammatory agent is selected from the group consisting essentially of ibuprofen, indomethacin, ketoprofen, flurbiprofen, celecoxib, valdecoxib, rofecoxib, varecoxib, parecoxib, meloxicam, nimesulide, etodolac, combinations and mixtures thereof.

17. The composition of claim 1, wherein said antifungal agent is selected from the classes of polyenes, allylamines, imidazoles, thiocarbamates, triazoles or combinations of these classes.

18. The composition of claim 1, wherein said antifungal agent is selected from amphotericin, nystatin, caspofungin, griseofulvin, oligomycins, butenafine, naftifine, terbinafine, bifonazole, clotrimazole, ketoconazole, miconazole, liranaftate, tolnaftate, fluconazole, itraconazole, or voriconazole.

19. The composition of claim 18, wherein the antifungal agent comprises butenafine.

20. The composition of claim 1, wherein the pharmaceutically acceptable vehicle further comprises a pH modifying agent selected from acids, bases, inorganic basic salts, organic basic salts, buffering agents or mixtures thereof.

21. The composition of claim 1, that is in a physical form selected from drops, pastes, ointments, creams, milks, pomades, powders, impregnated pads, tulles, solutions, gels, shampoos, lotions, suspensions, microspheres, nanospheres, lipidic vesicles, polymeric vesicles, polymeric patches or biological inserts.

22. A method of treating and/or preventing a bacterial infection disease comprising:
administering to a subject in need thereof, a pharmaceutical composition comprising:
a pharmaceutically effective amount of benzoquinolizine-2-carboxylic acid antimicrobial drug of the formula (I) according to claim 1; singly or in combination with a pharmaceutically effective amount(s) of a retinoid, an antibacterial, a steroid/non-steroid antiinflammatory agent, an antifungal agent or mixtures thereof.

23. The method of claim 33, wherein the benzoquinolizine-2-carboxylic acid antimicrobial drug is RS-(±)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof.

24. The method of claim 33, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid arginine salt and solvatomorphic or polymorphic forms thereof.

25. The method of claim 33, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid 0.2 hydrate.

26. The method of claim 33, wherein the benzoquinolizine-2-carboxylic acid antimicrobial drug is S-(−)-9-fluoro-6,7-dihydro-8-(4-hydroxypiperidin-1-yl)-5-methyl-1-oxo-1H,5H-benzo[i,j]quinolizine-2-carboxylic acid.

27. The method of claim 33, wherein the benzoquinolizine-2-carboxylic acid antimicrobial drug comprises about 0.1-10% by weight of the composition.

28. The method of claim 33, wherein the benzoquinolizine-2-carboxylic acid antimicrobial drug comprises about 1% by weight of the composition.

29. The method of claim 33, wherein the benzoquinolizine-2-carboxylic acid antimicrobial drug comprises about 0.5% by weight of the composition.

30. The method of claim 33, wherein the retinoid comprises adapalene.

31. The method of claim 33, wherein said antibacterial is selected from the classes of aminoglycosides, cephalosporins, diaminopyridines, oxazolidinones, sulfonamides, tetracyclines or combinations of these classes.

32. The method of claim 33, wherein the steroid comprises clobetasol.

33. The method of claim 33, wherein said non-steroid antiinflammatory agent is selected from the group consisting essentially of ibuprofen, indomethacin, ketoprofen, flurbiprofen, celecoxib, valdecoxib, rofecoxib, varecoxib, parecoxib, meloxicam, nimesulide, etodolac, combinations and mixtures thereof.

34. The method of claim 33, wherein the antifungal agent comprises butenafine.

35. The method of claim 33, wherein said composition is in a physical form selected from concentrates, drops, pastes, ointments, creams, milks, pomades, powders, impregnated pads, tulles, solutions, gels, sprays, shampoos, lotions, suspensions, microspheres, nanospheres, lipidic vesicles, polymeric vesicles, polymeric patches or biological inserts.

36. The method of claim 33, wherein the subject is an animal or human.

37. The method of claim 33, wherein the route of administration is selected from ocular, nasal, otic, rectal, vaginal, intradermal, intratumoral, intralesional, intravascular, topical, transdermal, local, regional, or loco-regional.
